(19) United States
(12) Patent Application Publication
(10) Pub. No.: US 2026/0272997 A1
(43) Pub. Date: Sep. 17, 2026

(54) Engineered skin commensal

(71) Applicant: The Regents of the University of California, Oakland, CA (US)

(72) Inventors: Amin Zargar, Berkeley, CA (US); Jay D. Keasling, Berkeley, CA (US); Adam M. Feist, Berkeley, CA (US); MyungHyun Noh, Berkeley, CA (US)

(73) Assignee: The Regents of the University of California, Oakland, CA (US)

(21) Appl. No.: 19/675,513

(22) Filed: May 13, 2026

Related U.S. Application Data
(63) Continuation of application No. PCT/US23/80182, filed on Nov. 17, 2023.

(51) Int. Cl. A61K 35/741; A61K 35/00; A61P 17/00

(52) U.S. Cl. CPC A61K 35/741; A61P 17/00; A61K 2035/115

(57) ABSTRACT

Mutant strains of Corynebacterium glutamicum bacteria are formulated as human skin commensals in compositions comprising a living population of engineered, mutant Corynebacterium glutamicum bacteria strain engineered to adopt characteristics of an optimized skin commensal bacteria.

### CROSS-REFERENCES TO RELATED APPLICATIONS

This application is a continuation of PCT/US23/80182, filed: Nov. 17, 2023, the disclosure of which is hereby incorporated by reference in its entirety for all purposes.

### INTRODUCTION

Genetically engineered probiotics that produce therapies on-site on the surface of the skin would be valuable for treating many diseases and also has cosmetic applications. We previously Corynebacterium glutamicum, a soil bacteria and Generally Recognized as Safe (GRAS) organism, as a probiotic to apply to topically to the skin (B21-024-2WO, PCT/US22/19051; WO2022/187724). We disclosed that C. glutamicum, when applied to the skin with a moisturizer that alters the microenvironment and provides nutrients, could colonize the skin for over 8 hours and produce glutamate on the surface of the skin. This was a novel and non-obvious idea as the soil microbe cannot natively grow on the skin surface.

### SUMMARY OF THE INVENTION

We disclose genetic engineering that enable C. glutamicum to adopt characteristics of an optimized skin commensal bacteria, namely the ability to grow in highly acidic and lipidic conditions. Application in a moisturizer that is also acidic prevents skin colonization by other harmful bacteria, and improved tolerance to lipids provides a more robust colonizer and expanded therapeutic options at the surface of the skin.

Culturing C. glutamicum for hundreds of generations in highly acidic and lipidic conditions that are considered “skin-mimicking” (pH~5.5 and Tween80~1%), we developed evolved strains that can grow rapidly in those conditions while the wild-type strain cannot grow in those conditions. The strains were whole-genome sequenced, and we identified mutations and amplifications that contribute to increased acid and lipid tolerance.

We show the growth rate of one of these strains to be 0.3 in “skin-mimicking” conditions, which is 60% of the growth rate in optimal rich media (0.5) and compares to the lack of exponential growth in the wild-type strain in “skin-mimicking” conditions.

The invention provides mutant strains of C. glutamicum, natively a soil microbe, optimized for colonizing skin. The strains may be further genetically engineered, such as disclosed in WO2022/187724, to produce therapies or cosmetic substances, such as for use in commercial applications in both health care and cosmetics.

In aspects and embodiments the invention provides:

1. A composition comprising a living population of engineered, mutant Corynebacterium glutamicum bacteria strain engineered to adopt characteristics of an optimized skin commensal bacteria, namely the ability to grow in acidic and lipidic conditions, such as pH 3.5-6.5, e.g. pH~5.5, and Tween80 of 0.2-5%, e.g. ~1%.

2. The composition of claim 1, wherein the mutant bacteria strain provides increased growth under acidic and lipidic condition compared with a corresponding wild-type strain.

3. The composition of claim 1, wherein the mutant bacteria strain is engineered to comprise a plurality of human skin commensal mutations or amplifications or Table 1, 2 or 3.

4. The composition of claim 1, wherein the mutant bacteria strain is engineered to comprise 1, 2, 3, 4, 5, 6, 7, 8 or 9 of genes: Ipps, cgp_3312, pmt, cgp_0753, atpB, atpE, cglR3, cgp3052 and cgp_r5123, and/or amplifications of 1, 2, 3, 4, 5, 6, 7, 8, 9, 10, 11, 12 or all of genes: cgp_1683, tatC, tatX, cgp_1686, cgp_1687, pafA2, pup, pafA1, mpa, pimT, pepC, cgp_1694, cgp_1695.

5. The composition of claim 1, wherein the mutant bacteria strain is engineered to comprise mutations and amplifications of Cgm-1A-1G or Cgm-2A-2E.

6. The composition of claim 1, 2, 3, 4 or 5, configured as a topical, human skin probiotic moisturizer formulation further comprising a nutrient source for the bacteria, wherein the formulation is acidic to prevent skin colonization by other harmful bacteria.

7. The composition of claim 6, disposed on the skin of a person.

8. The composition of claim 6, wherein the nutrient source comprises one or more sugars.

9. The composition of claim 6, further comprising a humectant, such as glycerin, hyaluronic acid, and propylene glycol, or an emollient, such as shea butter, cocoa butter, and octyldodecanol, or an occlusive, such as petrolatum, cetyl alcohol (hexadecan-1-ol), and lanolin.

10. The composition of claim 6, engineered to produce a skin-bioactive agent, wherein the bacteria are producing the agent.

11. The composition of claim 10, wherein the bioactive agent is selected from a cosmetic, anti-aging, anti-oxidant, sun-screen, anti-inflammatory, antimicrobial, analgesic, and therapeutic compound.

12. The composition of claim 10, wherein the bioactive agent is selected from signal peptides, carrier peptides, neurotransmitter inhibitor peptides, and enzyme inhibitor peptides.

13. The composition of claim 10, wherein the bioactive agent is selected from lysine, arginine, cysteine, histidine, alanine, serine, theronine, isoleucine, aspartic acid, valine, citrulline, GHK-Cu, GSH-Cu, mangenese tripeptide, Peptamide-6, carnosine, N-acetyl carnosine, tripeptide-10-citrulline, palmitoyl-tripeptides, palmitoyl-tetrapeptides, palmitoyl-pentapeptides, acetyl-tetrapeptide, hexapeptide-11, tetrapeptide PKEK, hexapeptide-14, silk protein, aquaporin, alpha interferon, Hsp70, transforming growth factors, rice peptides, and soybean peptides.

14. The composition of claim 10, wherein the bioactive agent is a serine protease inhibitor, such as LEKTI-D6, LEKTI-D5, elafin and secretory leukocyte protease inhibitor for treating Netherton Syndrome, atopic dermatitis, psoriasis, and acne rosacea.

15. The composition of claim 10, wherein the bioactive agent is an anti-inflammatory compound, such as IL-10 to treat diseases such as Netherton Syndrome, atopic dermatitis, psoriasis, acne rosacea, and chronic wound infection.

16. The composition of claim 10, wherein the bioactive agent is an anti-microbial peptide, such as pediocin and nisin to treat diseases such as Netherton Syndrome, atopic dermatitis, psoriasis, acne rosacea, and chronic wound infection.

17. The composition of claim 10, wherein the bioactive agent is an antibody, antibody fragment or nanobody, such as single chain variable fragment anti-tumor necrosis factor alpha to treat diseases such as Netherton Syndrome, atopic dermatitis, psoriasis, acne rosacea, and chronic wound infection.

18. A method of using composition of claim 6, comprising applying the formulation to the skin of a person in need thereof under conditions wherein the bacteria produce an effective amount of the agent on the skin over a predetermined time range.

19. A method of claim 18, wherein the time range is 4, 8, 12, 24, and 48 hours.

20. A method of claim 18, further comprising adjusting the concentration of the nutrient source to adjust bioproduction and colonization time, wherein the concentration of glucose and glycerol is varied from 2 to 20% of the moisturizer for increasingly longer colonization times.

The invention encompasses all combinations of the particular embodiments recited herein, as if each combination had been laboriously recited.

### BRIEF DESCRIPTION OF THE DRAWINGS

FIG. 1. Engineered C. glutamicum: Growth in Skin-Mimicking Media. The difference in growth between the original strain and one of the engineered strains. The original strain does not grow at all, while the engineered strain has a growth rate of 0.3. The growth rate in optimal rich media is 0.5, so the engineered strain has 60% of the growth strain in the high acid, lipidic environments of the skin-mimicking media as it does of the optimal rich media.

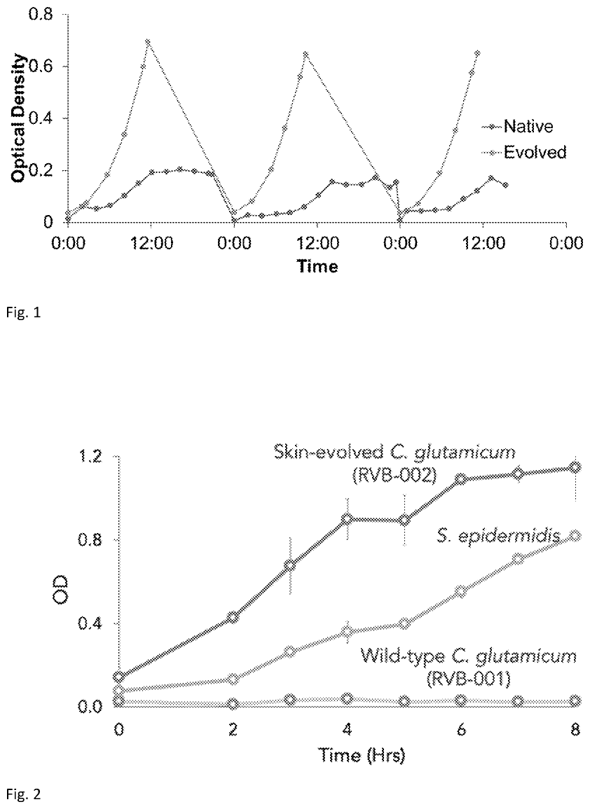

FIG. 2. Adaptive Laboratory Evolution of C. glutamicum to “skin-mimicking” (BHI, pH 5.5, and 1% Tween 80).

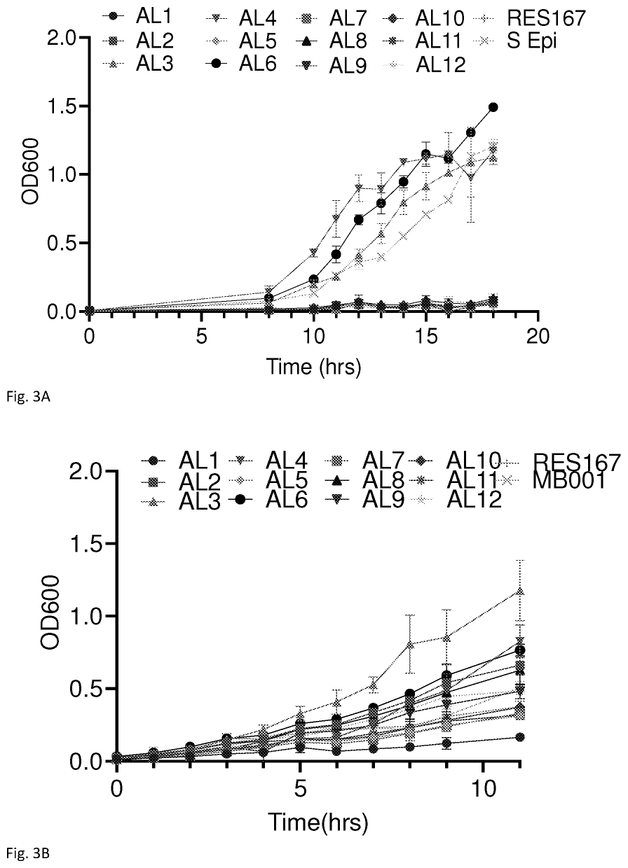

FIGS. 3A-D. Evaluate AL strains in different media conditions; Growth Rate is calculated as the slope of the LN of OD600 values during the growth phase. In all the different conditions, AL3, AL4 and AL6 had the best growth rates. A. Overnight in BHI, Overnight in Skin-BHI, Assay (OD~0.1) in tubes; B. Overnight in BHI, Exponential in BHI, Assay (OD~0.1) in tubes; C. Overnight in BHI, Overnight in Skin-BHI, Assay (OD~0.1) on plate; D. Overnight in BHI, Exponential in BHI, Assay (OD~0.1) on plate.

### DESCRIPTION OF PARTICULAR EMBODIMENTS OF THE INVENTION

Unless contraindicated or noted otherwise, in these descriptions and throughout this specification, the terms “a” and “an” mean one or more, the term “or” means and/or. The examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein, including citations therein, are hereby incorporated by reference in their entirety for all purposes.

### Examples. Safety and Efficacy of Applying to Skin a Probiotic Moisturizer Comprising the Soil Bacterium C. glutamicum Producing Glutamate

We used common moisturizer ingredients (paraffin oil, water, Span60, dimethicone, and stearic acid) with the needed components for C. glutamicum to grow (glucose, glycerol, yeast extract and biotin) so that C. glutamicum could produce nutrients on the skin surface. The moisturizer is combined in a 1:1 mixture with Corynebacterium glutamicum resuspended at a concentration of 10¹⁰ CFU/mL. Human testing was performed to determine both skin colonization of the soil bacterium and production of glutamate, an amino acid secreted and produced C. glutamicum. We used a control of moisturizer alone to determine local microbiome conditions. After 0, 2, 4, 8 and 24 hours we used a skin swab and determine the colony forming units in a 16 cm² portion on the skin. We determined that C. glutamicum stably colonizes the surface of the skin for 8 hours before dropping back to control conditions after 24 hours. We also found increasing and continuous production of C. glutamicum over 8 hours before dropping back to control conditions after 24 hours. Highlighting the power of C. glutamicum continuous production, a topical glutamate formula of 0.1% glutamate was added to the skin, and was undetectable after 15 minutes likely because of absorption and degradation on the skin surface, demonstrating the utility of bacterial produced compounds continuously on the surface. Our safety data from human trials also demonstrate that applying this organism on human skin is non-irritating and non-allergenic, confirming both safety and efficacy.

The acidity and sebum of the skin environment provides a defense mechanism against colonization by microbes. Adult skin pH measurements indicate that the pH of skin for women (mean pH=5.54) is slightly lower than men (mean pH=5.80)⁸. Therefore, we sought to engineer our strain to grow in a “skin mimicking” medium that is highly acidic (pH~5.5) and contains a sebum analog (1% Tween 80).

We conducted a 45-day Adaptive Laboratory Evolution study on seven clonal isolates of C. glutamicum, gradually decreasing the pH and increasing the sebum analog over thousands of generations. The genomes of resultant strains of C. glutamicum which exhibited robust growth under skin-mimicking conditions were then sequenced. Our results demonstrate that wild-type C. glutamicum was unable to grow under these conditions, while S. epidermidis, a commensal skin bacterium, exhibited a growth rate of 0.29. Remarkably, our evolved C. glutamicum strain (RVB-002) showcased a growth rate of 0.47, surpassing S. epidermidis by 62%. This successful evolution of C. glutamicum demonstrates its potential to temporarily colonize skin environments to produce targeted therapeutic delivery.

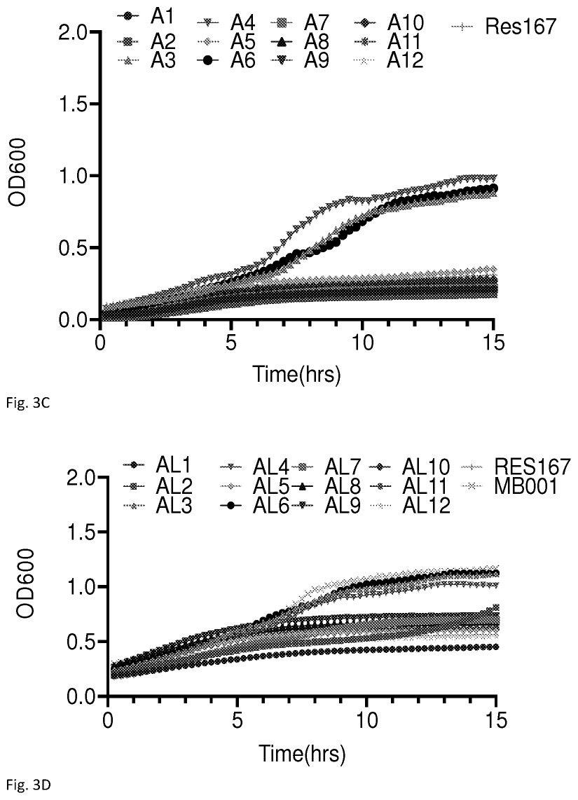

We tested our evolved strains in a variety of conditions. We tested four conditions for all of our strains. In FIG. 3A, we grew our strains in skin-mimicking conditions, passaged them again in skin-mimicking conditions, then grew the strains to OD~0.1 before measuring the growth rate in skin-mimicking media in test tubes for 18 hours. We found that some of our evolved strains grew even better than the most common skin microbe, S. epidermidis. In FIG. 3C, we performed the same experiment, but this time on microtiter plates. In FIG. 3B, we grew our strains in skin-mimicking conditions, grew them to exponential phase in rich BHI, then diluted the strains to OD~0.1 before measuring the growth rate in skin-mimicking media in test tubes for 12 hours. In FIG. 3D, we performed the same experiment, but in microtiter plates. Across all experiments, AL3, AL4, and AL6 had the highest growth rates. These strains all include amplications of these genes: cgp_1683, tatC, tatX, cgp_1686, cgp_1687, pafA2, pup, pafA1, mpa, pimT, pepC, cgp_1694, cgp_1695, indicating that these genes contribute towards this phenotype.

TABLE 1
Exemplary C. glutamicum mutant strains providing
improved growth on human skin compared to wild-type strain.
Cgm-1A

| | | |
|---|---|---|
| SNP | cgp_0001 | P420R (CCA > CGA) |
| INS | cgp_0790, pyc | intergenic (+217/−180) |
| INS | cgp_0790, pyc | intergenic (+223/−174) |
| INS | lppS | coding (414/1212 nt) |
| INS | lppS | coding (410/1212 nt) |

Genetic Amplications: Sequence Change/Genes
50,800 bp x 2
cgp_1663, cgp_1664, cgp_1665, cgp_1668, cgp_1669, cgp_1670, cgp_1671, ppmC, ppmN, cgp_1675,
lip3, cgp_1677, cobL, cgp_4020, cgp_1679, cgp_1680, pepE, cgp_1682, cgp_1683, tatC, tatX, cgp_1686,
cgp_1687, pafA2, pup, pafA1, mpa, pimT, pepC, cgp_1694, cgp_1695, cgp_1696, cgp_1697, hisG,
hisE, cgp_1700, metH, cgp_1702, cgp_1703, cgp_1704, arsC2, cgp_1706, cgp_1707, cgp_1708, mshC,
bacA, cgp_1711, lppL, pyrD, cgp_1714
Cgm-1B

| | | |
|---|---|---|
| SNP | mag | G94S (GGT > AGT) |
| SNP | cgp_1121, cgp_1122 | intergenic (+341/+109) |
| SNP | cgp_1275 | E97K (GAA > AAA) |
| SNP | tatX | W6S (TGG > TCG) |
| SNP | clpC, guaB1 | intergenic (−8/−281) |
| SNP | cgp_0811 | T219A (ACC > GCC) |
| SNP | mgtE1 | A176T (GCC > ACC) |
| SNP | lppS | E230* (GAA > TAA) |
| SNP | cgp_2756 | T139R (ACA > AGA) |

Cgm-1C

| | | |
|---|---|---|
| SNP | cgp_0811 | T219A (ACC > GCC) |
| SNP | mgtE1 | A176T (GCC > ACC) |
| SNP | lppS | E230* (GAA > TAA) |
| SNP | cgp_2756 | T139R (ACA > AGA) |

Genetic Amplications: Sequence Change/Genes
83,700 bp x
cgp_1663, cgp_1664, cgp_1665, cgp_1668, cgp_1669, cgp_1670, cgp_1671, ppmC, ppmN, cgp_1675,
lip3, cgp_1677, cobL, cgp_4020, cgp_1679, cgp_1680, pepE, cgp_1682, cgp_1683, tatC, tatX, cgp_1686,
cgp_1687, pafA2, pup, pafA1, mpa, pimT, pepC, cgp_1694, cgp_1695, cgp_1696, cgp_1697, hisG,
hisE, cgp_1700, metH, cgp_1702, cgp_1703, cgp_1704, arsC2, cgp_1706, cgp_1707, cgp_1708, mshC,
bacA, cgp_1711, lppL, pyrD, cgp_1714, cgp_1715, cgp_1716, cgp_1717, cgp_1718, tetB, tetA, cgp_t5033,
cgp_t5034, cgp_1722, cgp_1724, mcmB, mcmA, cgp_1727, cgp_1728, cgp_1730, cgp_1731,
cgp_1732, cgp_1733, hemH, cgp_1735, cgp_1736, acn, acnR, cgp_1739, cgp_1740, cgp_1741,
cgp_1742, cgp_1743, pacL
Cgm-1D

| | | |
|---|---|---|
| SNP | cgp_0569 | T281P (ACC > CCC) |
| INS | cgp_1159 | coding (1757/1905 nt) |
| SNP | atpA | P291L (CCA > CTA) |
| SNP | cgp_2535 | A136A (GCC > GCT) |
| DEL | cgp_3312 | coding (307/1461 nt) |

Genetic Amplications: Sequence Change/Genes
50,800 bp x
3cgp_1663, cgp_1664, cgp_1665, cgp_1668, cgp_1669, cgp_1670, cgp_1671, ppmC, ppmN, cgp_1675,
lip3, cgp_1677, cobL, cgp_4020, cgp_1679, cgp_1680, pepE, cgp_1682, cgp_1683, tatC, tatX, cgp_1686,
cgp_1687, pafA2, pup, pafA1, mpa, pimT, pepC, cgp_1694, cgp_1695, cgp_1696, cgp_1697, hisG,
hisE, cgp_1700, metH, cgp_1702, cgp_1703, cgp_1704, arsC2, cgp_1706, cgp_1707, cgp_1708, mshC,
bacA, cgp_1711, lppL, pyrD, cgp_1714
Cgm-1E

| | | |
|---|---|---|
| DEL | pmt | coding (350/1563 nt) |
| SNP | atpD | D322Y (GAT > TAT) |
| SNP | cgtR3 | D187N (GAT > AAT) |

Cgm-1F

| | | |
|---|---|---|
| SNP | cgp_0001 | D509H (GAT > CAT) |
| SNP | cmt5 | E188Q (GAA > CAA) |
| DEL | lppS | coding (1146/1212 nt) |
| SNP | glpQ1, gntP | intergenic (−139/−283) |
| SNP | cgp_3348, nagL | intergenic (−284/+254) |

Genetic Amplications: Sequence Change/Genes
12,600 bp x 2cgp_1683, tatC, tatX, cgp_1686, cgp_1687, pafA2, pup, pafA1, mpa, pimT, pepC,
cgp_1694, cgp_1695
Cgm-1G

| | | |
|---|---|---|
| INS | cgp_1159 | coding (1764/1905 nt) |
| INS | cgp_1159 | coding (1757/1905 nt) |

Genetic Amplications: Sequence Change/Genes
14,100 bp x 3cgp_1680, pepE, cgp_1682, cgp_1683, tatC, tatX, cgp_1686, cgp_1687, pafA2, pup,
pafAl, mpa, pimT
Cgm-2A

| | | |
|---|---|---|
| SNP | atpB, atpE | intergenic (+99/−35) |
| SNP | cgp_0666 | T192A (ACT→GCT) |
| SNP | cgp_0753 | G159E (GGA→GAA) |
| SUB | cgp_6007, psp2 | intergenic (+337/−311) |
| DEL | pmt | coding (350/1563 nt) |
| SNP | rpoC | C441Y (TGT→TAT) |
| INS | sufR, cgp_1766 | intergenic (−88/−234) |

Cgm-2B

| | | |
|---|---|---|
| SNP | atpG | G314R (GGC→CGC) |
| INS | cgp_0223, tnp2f | intergenic (−658/−119) |
| DEL | cgp_0753 | coding (178-201/966 nt) |
| DEL | cgp_1016a | coding (334/360 nt) |
| SNP | cgp_1103 | M82I (ATG→ATC) |
| DEL | cgp_1467, cgp_1468 | intergenic (−65/+19) |
| DEL | cgp_2380 | coding (270/390 nt) |
| DEL | metY, cstA | intergenic (−354/−66) |
| INS | pmt | coding (350/1563 nt) |
| INS | pmt | coding (356/1563 nt) |

Genetic Amplications: Sequence Change/Genes
57,900 bp x
4cgp_1680, pepE, cgp_1682, cgp_1683, tatC, tatX, cgp_1686, cgp_1687, pafA2, pup, pafA1, mpa, pimT,
pepC, cgp_1694, cgp_1695, cgp_1696, cgp_1697, hisG, hisE, cgp_1700, metH, cgp_1702, cgp_1703,
cgp_1704, arsC2, cgp_1706, cgp_1707, cgp_1708, mshC, bacA, cgp_1711, lppL, pyrD, cgp_1714,
cgp_1715, cgp_1716, cgp_1717, cgp_1718, tetB, tetA, cgp_t5033, cgp_t5034, cgp_1722, cgp_1724,
mcmB, mcmA, cgp_1727, cgp_1728, cgp_1730, cgp_1731, cgp_1732, cgp_1733, hemH
Cgm-2C

| | | |
|---|---|---|
| SNP | cgp_0811 | T219A (ACC→GCC) |
| SNP | cgp_1288 | W105* (TGG→TAG) |
| SNP | cgp_2756 | T139R (ACA→AGA) |
| SNP | cgp_3084 | E221E (GAG→GAA) |
| SNP | dtxR | P127L (CCT→CTT) |
| SNP | lppS | E230* (GAA→TAA) |

Cgm-2D

| | | |
|---|---|---|
| SNP | atpA | P292R (CCG→CGG) |
| SNP | cgp_2810, cgp_2811 | intergenic (−139/+196) |
| DEL | cgp_2854 | Δ1,639 bp |
| SUB | cgp_6007, psp2 | intergenic (+337/−311) |
| SNP | cgtR3 | D187N (GAT→AAT) |
| INS | mgtE1 | coding (742/1293 nt) |
| INS | pmt | coding (350/1563 nt) |

Genetic Amplications: Sequence Change/Genes
12,500 bp x 2cgp_1683, tatC, tatX, cgp_1686, cgp_1687, pafA2, pup, pafA1, mpa, pimT, pepC,
cgp_1694, cgp_1695
Cgm-2E

| | | |
|---|---|---|
| INS | cgp_0223, tnp2f | intergenic (−658/−119) |
| DEL | cgp_1016a | coding (334/360 nt) |
| DEL | cgp_1467, cgp_1468 | intergenic (−65/+19) |
| DEL | cgp_2380 | coding (270/390 nt) |
| SNP | cgp_2737 | P120Q (CCG→CAG) |
| DEL | cgp_2854 | Δ1,639 bp |
| SUB | cgp_6007, psp2 | intergenic (+337/−311) |
| SNP | cgtR3 | N188K (AAC→AAG) |
| DEL | metY, cstA | intergenic (−354/−66) |
| INS | pmt | coding (350/1563 nt) |
| INS | pmt | coding (356/1563 nt) |
| SNP | rpsA | N219D (AAC→GAC) |
| SNP | rpsH | G97G (GGC→GGT) |

TABLE 2

| | | | | | | |
|---|---|---|---|---|---|---|
| Reference | | Mutation | Sequence | Gene | | |
| Seq | Position | Type | Change | (Scrollable) | Product | Details |

MutationsRES167

| | | | | | | |
|---|---|---|---|---|---|---|
| CP005959 | 1,278,778 | SNP | C→G | atpA | ATP synthase F1, alpha | P292R |
| | | | | | chain | (CCG→CGG) |
| CP005959 | 1,276,147 | SNP | C→G | atpB, | ATP synthase F0, A chain/ | intergenic |
| | | | | atpE | ATP synthase F0, C chain | (+99/−35) |
| CP005959 | 1,276,111 | SNP | G→A | atpB, | ATP synthase F0, A chain/ | intergenic |
| | | | | atpE | ATP synthase F0, C chain | (+63/−71) |
| CP005959 | 1,280,543 | SNP | G→C | atpG | ATP synthase F1, gamma | G314R |
| | | | | | chain | (GGC→CGC) |
| CP005959 | 192,812 | INS | +C | cgp_0223, | putative sugar/metabolite | intergenic |
| | | | | tnp2f | permease, MFS- | (−658/−119) |
| | | | | | type/transposase | |
| CP005959 | 589,872 | SNP | T→C | cgp_0666 | putative membrane protein | T192A |
| | | | | | | (ACT→GCT) |
| CP005959 | 667,495 | SNP | C→T | cgp_0753 | putative secreted protein | G159E |
| | | | | | | (GGA→GAA) |
| CP005959 | 667,770 | DEL | Δ24 bp | cgp_0753 | putative secreted protein | coding |
| | | | | | | (178-201/966 nt) |
| CP005959 | 730,592 | SNP | T→C | cgp_0811 | acetyl/propionyl-CoA | T219A |
| | | | | | carboxylase, beta chain | (ACC→GCC) |
| CP005959 | 948,224 | DEL | Δ1 bp | cgp_1016a | | coding |
| | | | | | | (334/360 nt) |
| CP005959 | 1,031,651 | SNP | C→G | cgp_1103 | putative membrane protein | M82I |
| | | | | | | (ATG→ATC) |
| CP005959 | 1,189,034 | SNP | G→A | cgp_1288 | putative multidrug efflux | W105* |
| | | | | | permease, MFS-type | (TGG→TAG) |
| CP005959 | 1,275,104 | SNP | G→A | cgp_1361, | hypothetical protein/ATP | intergenic |
| | | | | atpB | synthase F0, A chain | (+86/−9) |
| CP005959 | 1,367,753 | DEL | Δ1 bp | cgp_1467, | putative transcriptional | intergenic |
| | | | | cgp_1468 | regulator, TetR- | (−65/+19) |
| | | | | | family/hypothetical protein | |
| CP005959 | 2,064,843 | DEL | Δ1 bp | cgp_2380 | putative membrane protein | coding |
| | | | | | | (270/390 nt) |
| CP005959 | 2,402,715 | SNP | C→A | cgp_2737 | putative transcriptional | P120Q |
| | | | | | regulator, TetR-family | (CCG→CAG) |
| CP005959 | 2,421,817 | SNP | G→C | cgp_2756 | hypothetical protein | T139R |
| | | | | | | (ACA→AGA) |
| CP005959 | 2,467,941 | SNP | G→T | cgp_2810, | putative secondary | intergenic |
| | | | | cgp_2811 | H+/Na+:glutamate/ | (−139/+196) |
| | | | | | dicarboxylate symporter, | |
| | | | | | dicarboxylate/amino | |
| | | | | | acid:cation symporter | |
| | | | | | (DAACS) family/ABC-type | |
| | | | | | lipoprotein release | |
| | | | | | transporter, permease | |
| | | | | | subunit | |
| CP005959 | 2,512,825 | DEL | Δ1,639 bp | cgp_2854 | cgp_2854 | |
| CP005959 | 2,712,200 | SNP | T→C | cgp_3052 | putative secreted protein | T52A |
| | | | | | | (ACA→GCA) |
| CP005959 | 2,740,349 | SNP | G→A | cgp_3084 | putative flavoprotein | E221E |
| | | | | | involved in K+ transport | (GAG→GAA) |
| CP005959 | 839,894 | SUB | 2 bp→AA | cgp_6007, | hypothetical protein/putative | intergenic |
| | | | | psp2 | secreted protein | (+337/−311) |
| CP005959 | 2,544,109 | SNP | C→T | cgtR3 | two-component system, | D187N |
| | | | | | transcriptional response | (GAT→AAT) |
| | | | | | regulator | |
| CP005959 | 2,544,104 | SNP | G→C | cgtR3 | two-component system, | N188K |
| | | | | | transcriptional response | (AAC→AAG) |
| | | | | | regulator | |
| CP005959 | 1,789,237 | SNP | C→T | dtxR | transcriptional regulator, | P127L |
| | | | | | DtxR-family | (CCT→CTT) |
| CP005959 | 2,389,708 | SNP | C→A | IppS | putative secreted | E230* |
| | | | | | lipoprotein, | (GAA→TAA) |
| | | | | | ErfK/YbiS/YcfS/YnhG-family | |
| CP005959 | 671,006 | DEL | Δ13 bp | metY, | O-Acetylhomoserine | intergenic |
| | | | | cstA | sulfhydrylase/putative | (−354/−66) |
| | | | | | carbon starvation protein A | |
| CP005959 | 1,172,765 | INS | (ATC)2→3 | mgtE1 | putative Mg2+ transporter, | coding |
| | | | | | Mg2+ transporter-E (MgtE) | (742/1293 nt) |
| | | | | | family | |
| CP005959 | 946,486 | DEL | (G)6→5 | pmt | protein O- | coding |
| | | | | | mannosyltransferase | (350/1563 nt) |
| CP005959 | 946,486 | INS | (G)6→7 | pmt | protein O- | coding |
| | | | | | mannosyltransferase | (350/1563 nt) |
| CP005959 | 946,480 | INS | +G | pmt | protein O- | coding |
| | | | | | mannosyltransferase | (356/1563 nt) |
| CP005959 | 517,775 | SNP | G→A | rpoC | DNA-directed RNA | C441Y |
| | | | | | polymerase, beta′ chain | (TGT→TAT) |
| CP005959 | 1,411,684 | SNP | A→G | rpsA | 30S ribosomal protein S1 | N219D |
| | | | | | | (AAC→GAC) |
| CP005959 | 557,048 | SNP | C→T | rpsH | 30S ribosomal protein S8 | G97G |
| | | | | | | (GGC→GGT) |
| CP005959 | 1,639,449 | INS | (C)6→7 | sufR, | transcriptional repressor of | intergenic |
| | | | | cgp_1766 | suf operon/putative | (−88/−234) |
| | | | | | membrane protein | |
| CP005959 | 2,512,825 | DEL | Δ1,639 bp | cgp_2854 | cgp_2854 | |
| CP005959 | 839,894 | SUB | 2 bp→AA | cgp_6007, | hypothetical protein/putative | intergenic |
| | | | | psp2 | secreted protein | (+337/−311) |
| CP005959 | 2,544,104 | SNP | G→C | cgtR3 | two-component system, | N188K |
| | | | | | transcriptional response | (AAC→AAG) |
| | | | | | regulator | |
| CP005959 | 671,006 | DEL | Δ13 bp | metY, | O-Acetylhomoserine | intergenic |
| | | | | cstA | sulfhydrylase/putative | (−354/−66) |
| | | | | | carbon starvation protein A | |
| CP005959 | 946,486 | INS | (G)6→7 | pmt | protein O- | coding |
| | | | | | mannosyltransferase | (350/1563 nt) |
| CP005959 | 946,480 | INS | +G | pmt | protein O- | coding |
| | | | | | mannosyltransferase | (356/1563 nt) |
| CP005959 | 1,411,684 | SNP | A→G | rpsA | 30S ribosomal protein S1 | N219D |
| | | | | | | (AAC→GAC) |
| CP005959 | 557,048 | SNP | C→T | rpsH | 30S ribosomal protein S8 | G97G |
| | | | | | | (GGC→GGT) |

MutationsMB0015

| | | | | | | |
|---|---|---|---|---|---|---|
| CP005959 | 1,259 | SNP | C > G | cgp_0001 | chromosomal replication | P420R |
| | | | | | initiator protein DnaA | (CCA > CGA) |
| CP005959 | 1,525 | SNP | G > C | cgp_0001 | chromosomal replication | D509H |
| | | | | | initiator protein DnaA | (GAT > CAT) |
| CP005959 | 21,623 | SNP | A > T | cgp_0034 | | A16A |
| | | | | | | (GCA > GCT) |
| CP005959 | 129,755 | SNP | C > T | mag | putative 3-methylpurine DNA | G94S |
| | | | | | glycosylase | (GGT > AGT) |
| CP005959 | 506,537 | SNP | A > C | cgp_0569 | putative Cd2+ transporting P- | T281P |
| | | | | | type ATPase | (ACC > CCC) |
| CP005959 | 707,925 | INS | +G | cgp_0790, | flavoprotein disulfid reductase | intergenic |
| | | | | pyc | family protein/pyruvate | (+217/−180) |
| | | | | | carboxylase | |
| CP005959 | 707,931 | INS | (G)6 > 7 | cgp_0790, | flavoprotein disulfid reductase | intergenic |
| | | | | pyc | family protein/pyruvate | (+223/−174) |
| | | | | | carboxylase | |
| CP005959 | 730,592 | SNP | T > C | cgp_0811 | acetyl/propionyl-CoA | T219A |
| | | | | | carboxylase, beta chain | (ACC > GCC) |
| CP005959 | 946,486 | DEL | (G)6 > 5 | pmt | protein O- | coding |
| | | | | | mannosyltransferase | (350/1563 nt) |
| CP005959 | ####### | SNP | C > T | cgp_1121, | permease, MFS-type/putative | intergenic |
| | | | | cgp_1122 | secreted protein | (+341/+109) |
| CP005959 | ####### | INS | +G | cgp_1159 | putative secreted protein | coding |
| | | | | | | (1764/1905 nt) |
| CP005959 | ####### | INS | (G)7 > 8 | cgp_1159 | putative secreted protein | coding |
| | | | | | | (1757/1905 nt) |
| CP005959 | ####### | SNP | C > G | cmt5 | corynomycolyl transferase | E188Q |
| | | | | | | (GAA > CAA) |
| CP005959 | ####### | SNP | C > T | cgp_1275 | putative membrane protein | E97K |
| | | | | | | (GAA > AAA) |
| CP005959 | ####### | SNP | C > T | mgtE1 | putative Mg2+ transporter, | A176T |
| | | | | | Mg2+ transporter-E (MgtE) | (GCC > ACC) |
| | | | | | family | |
| CP005959 | ####### | SNP | G > A | cgp_1361, | hypothetical protein/ATP | intergenic |
| | | | | atpB | synthase F0, A chain | (+86/−9) |
| CP005959 | ####### | SNP | C > T | atpA | ATP synthase F1, alpha chain | P291L |
| | | | | | | (CCA > CTA) |
| CP005959 | ####### | SNP | G > T | atpD | ATP synthase F1, beta chain | D322Y |
| | | | | | | (GAT > TAT) |
| CP005959 | ####### | SNP | C > G | tatX | putative twin arginine | W6S |
| | | | | | targeting (Tat) Preprotein | (TGG > TCG) |
| | | | | | translocase subunit | |
| CP005959 | ####### | SNP | G > A | cgp_2535 | putative secreted protein | A136A |
| | | | | | | (GCC > GCT) |
| CP005959 | ####### | SNP | G > A | cgp_2675 | ABC-type putative | T424T |
| | | | | | dipeptide/oligopeptide | (ACC > ACT) |
| | | | | | transporter, ATPase subunit | |
| CP005959 | ####### | DEL | (C)5 > 4 | IppS | putative secreted lipoprotein, | coding |
| | | | | | ErfK/YbiS/YcfS/YnhG-family | (1146/1212 nt) |
| CP005959 | ####### | SNP | C > A | IppS | putative secreted lipoprotein, | E230* |
| | | | | | ErfK/YbiS/YcfS/YnhG-family | (GAA > TAA) |
| CP005959 | ####### | INS | +G | lppS | putative secreted lipoprotein, | coding |
| | | | | | ErfK/YbiS/YcfS/YnhG-family | (414/1212 nt) |
| CP005959 | ####### | INS | +G | lppS | putative secreted lipoprotein, | coding |
| | | | | | ErfK/YbiS/YcfS/YnhG-family | (410/1212 nt) |
| CP005959 | ####### | SNP | G > C | cgp_2756 | hypothetical protein | T139R |
| | | | | | | (ACA > AGA) |
| CP005959 | ####### | SNP | C > T | cgtR3 | two-component system, | D187N |
| | | | | | transcriptional response | (GAT > AAT) |
| | | | | | regulator | |
| CP005959 | ####### | SNP | C > G | clpC, | putative ATP-dependent | intergenic |
| | | | | guaB1 | protease (heat shock | (−8/−281) |
| | | | | | protein)/IMP dehydrogenase | |
| CP005959 | ####### | SNP | C > T | glpQ1, | glycerophosphodiester | intergenic |
| | | | | gntP | phosphodiesterase/putative | (−139/−283) |
| | | | | | secondary gluconate | |
| | | | | | symporter, gluconate:H+ | |
| | | | | | symporter (GntP) family | |
| CP005959 | ####### | DEL | (C)6 > 5 | cgp_3312 | putative membrane protein | coding |
| | | | | | | (307/1461 nt) |
| CP005959 | ####### | SNP | C > T | cgp_3348, | putative plasmid maintenance | intergenic |
| | | | | nagL | system antidote | (−284/+254) |
| | | | | | protein/maleylpyruvate | |
| | | | | | isomerase, GSH-independent | |

TABLE 3

| | | | | | |
|---|---|---|---|---|---|
| Reference | | Mutation | Sequence | | |
| Seq | Position | Type | Change | Gene (Scrollable) | Details |

AmplifcationRES167

| | | | | | |
|---|---|---|---|---|---|
| CP005959 | 1,554,801 | AMP | 57,900 | cgp_1680, pepE, cgp_1682, cgp_1683, tatC, | amplification |
| | | | bp × 4 | tatX, cgp_1686, cgp_1687, pafA2, pup, | |
| | | | | pafA1, mpa, pimT, pepC, cgp_1694, | |
| | | | | cgp_1695, cgp_1696, cgp_1697, hisG, hisE, | |
| | | | | cgp_1700, metH, cgp_1702, cgp_1703, | |
| | | | | cgp_1704, arsC2, cgp_1706, cgp_1707, | |
| | | | | cgp_1708, mshC, bacA, cgp_1711, lppL, pyrD, | |
| | | | | cgp_1714, cgp_1715, cgp_1716, cgp_1717, | |
| | | | | cgp_1718, tetB, tetA, cgp_t5033, cgp_t5034, | |
| | | | | cgp_1722, cgp_1724, mcmB, mcmA, | |
| | | | | cgp_1727, cgp_1728, cgp_1730, cgp_1731, | |
| | | | | cgp_1732, cgp_1733, hemH | |
| CP005959 | 1,559,601 | AMP | 12,500 | cgp_1683, tatC, tatX, cgp_1686, cgp_1687, | duplication |
| | | | bp × 2 | pafA2, pup, pafA1, mpa, pimT, pepC, | |
| | | | | cgp_1694, cgp_1695 | |

AmplifcationMB001

| | | | | | |
|---|---|---|---|---|---|
| CP00559 | 91,541,501 | AMP | 50,800 | cgp_1663, cgp_1664, cgp_1665, cgp_1668, | duplication |
| | | | bp × 2 | cgp_1669, cgp_1670, cgp_1671, ppmC, | |
| | | | | ppmN, cgp_1675, lip3, cgp_1677, cobL, | |
| | | | | cgp_4020, cgp_1679, cgp_1680, pepE, | |
| | | | | cgp_1682, cgp_1683, tatC, tatX, cgp_1686, | |
| | | | | cgp_1687, pafA2, pup, pafA1, mpa, pimT, | |
| | | | | pepC, cgp_1694, cgp_1695, cgp_1696, | |
| | | | | cgp_1697, hisG, hisE, cgp_1700, metH, | |
| | | | | cgp_1702, cgp_1703, cgp_1704, arsC2, | |
| | | | | cgp_1706, cgp_1707, cgp_1708, mshC, | |
| | | | | bacA, cgp_1711, lppL, pyrD, cgp_1714 | |
| CP005959 | 1,541,501 | AMP | 50,800 | cgp_1663, cgp_1664, cgp_1665, cgp_1668, | amplification |
| | | | bp × 3 | cgp_1669, cgp_1670, cgp_1671, ppmC, | |
| | | | | ppmN, cgp_1675, lip3, cgp_1677, cobL, | |
| | | | | cgp_4020, cgp_1679, cgp_1680, pepE, | |
| | | | | cgp_1682, cgp_1683, tatC, tatX, cgp_1686, | |
| | | | | cgp_1687, pafA2, pup, pafA1, mpa, pimT, | |
| | | | | pepC, cgp_1694, cgp_1695, cgp_1696, | |
| | | | | cgp_1697, hisG, hisE, cgp_1700, metH, | |
| | | | | cgp_1702, cgp_1703, cgp_1704, arsC2, | |
| | | | | cgp_1706, cgp_1707, cgp_1708, mshC, | |
| | | | | bacA, cgp_1711, lppL, pyrD, cgp_1714 | |
| CP005959 | 1,541,601 | AMP | 83,700 | cgp_1663, cgp_1664, cgp_1665, cgp_1668, | amplification |
| | | AMP | bp × 5 | cgp_1669, cgp_1670, cgp_1671, ppmC, | |
| | | | | ppmN, cgp_1675, lip3, cgp_1677, cobL, | |
| | | | | cgp_4020, cgp_1679, cgp_1680, pepE, | |
| | | | | cgp_1682, cgp_1683, tatC, tatX, cgp_1686, | |
| | | | | cgp_1687, pafA2, pup, pafA1, mpa, pimT, | |
| | | | | pepC, cgp_1694, cgp_1695, cgp_1696, | |
| | | | | cgp_1697, hisG, hisE, cgp_1700, metH, | |
| | | | | cgp_1702, cgp_1703, cgp_1704, arsC2, | |
| | | | | cgp_1706, cgp_1707, cgp_1708, mshC, | |
| | | | | bacA, cgp_1711, lppL, pyrD, cgp_1714, | |
| | | | | cgp_1715, cgp_1716, cgp_1717, cgp_1718, | |
| | | | | tetB, tetA, cgp_t5033, cgp_t5034, cgp_1722, | |
| | | | | cgp_1724, mcmB, mcmA, cgp_1727, | |
| | | | | cgp_1728, cgp_1730, cgp_1731, cgp_1732, | |
| | | | | cgp_1733, hemH, cgp_1735, cgp_1736, | |
| | | | | acn, acnR, cgp_1739, cgp_1740, cgp_1741, | |
| | | | | cgp_1742, cgp_1743, pacL | |
| CP005959 | 1,541,601 | | 83,700 | cgp_1663, cgp_1664, cgp_1665, cgp_1668, | amplification |
| | | | bp × 4 | cgp_1669, cgp_1670, cgp_1671, ppmC, | |
| | | | | ppmN, cgp_1675, lip3, cgp_1677, cobL, | |
| | | | | cgp_4020, cgp_1679, cgp_1680, pepE, | |
| | | | | cgp_1682, cgp_1683, tatC, tatX, cgp_1686, | |
| | | | | cgp_1687, pafA2, pup, pafA1, mpa, pimT, | |
| | | | | pepC, cgp_1694, cgp_1695, cgp_1696, | |
| | | | | cgp_1697, hisG, hisE, cgp_1700, metH, | |
| | | | | cgp_1702, cgp_1703, cgp_1704, arsC2, | |
| | | | | cgp_1706, cgp_1707, cgp_1708, mshC, | |
| | | | | bacA, cgp_1711, lppL, pyrD, cgp_1714, | |
| | | | | cgp_1715, cgp_1716, cgp_1717, cgp_1718, | |
| | | | | tetB, tetA, cgp_t5033, cgp_t5034, cgp_1722, | |
| | | | | cgp_1724, mcmB, mcmA, cgp_1727, | |
| | | | | cgp_1728, cgp_1730, cgp_1731, cgp_1732, | |
| | | | | cgp_1733, hemH, cgp_1735, cgp_1736, | |
| | | | | acn, acnR, cgp_1739, cgp_1740, cgp_1741, | |
| | | | | cgp_1742, cgp_1743, pacL | |
| CP005959 | 1,555,201 | AMP | 14,100 | cgp_1680, pepE, cgp_1682, cgp_1683, | amplification |
| | | | bp × 3 | tatC, tatX, cgp_1686, cgp_1687, pafA2, pup, | |
| | | | | pafA1, mpa, pimT | |
| CP005959 | 1,556,601 | AMP | 13,000 | cgp_1682, cgp_1683, tatC, tatX, cgp_1686, | amplification |
| | | | bp × 3 | cgp_1687, pafA2, pup, pafA1, mpa, pimT, | |
| | | | | pepC | |
| CP005959 | 1,559,501 | AMP | 12,600 | cgp_1683, tatC, tatX, cgp_1686, cgp_1687, | duplication |
| | | | bp × 2 | pafA2, pup, pafA1, mpa, pimT, pepC, | |
| | | | | cgp_1694, cgp_1695 | |
| CP005959 | 1,559,601 | AMP | 12,500 | cgp_1683, tatC, tatX, cgp_1686, cgp_1687, | duplication |
| | | | bp × 2 | pafA2, pup, pafA1, mpa, pimT, pepC, | |
| | | | | cgp_1694, cgp_1695 | |

1. A composition comprising a living population of engineered, mutant Corynebacterium glutamicum bacteria strain engineered to adopt characteristics of an optimized skin commensal bacteria, namely the ability to grow in acidic and lipidic conditions, such as pH 3.5-6.5, e.g. pH~5.5, and Tween80 of 0.2-5%, e.g. ~1%.

2. The composition of claim 1, wherein the mutant bacteria strain provides increased growth under acidic and lipidic condition compared with a corresponding wild-type strain.

3. The composition of claim 1, wherein the mutant bacteria strain is engineered to comprise a plurality of human skin commensal mutations or amplifications or Table 1, 2 or 3.

4. The composition of claim 1, wherein the mutant bacteria strain is engineered to comprise 1, 2, 3, 4, 5, 6, 7, 8 or 9 of genes: 1pps, cgp_3312, pmt, cgp_0753, atpB, atpE, cglR3, cgp3052 and cgp_r5123, and/or amplifications of 1, 2, 3, 4, 5, 6, 7, 8, 9, 10, 11, 12 or all of genes: cgp_1683, tatC, tatX, cgp_1686, cgp_1687, pafA2, pup, pafA1, mpa, pimT, pepC, cgp_1694, cgp_1695.

5. The composition of claim 1, wherein the mutant bacteria strain is engineered to comprise mutations and amplifications of Cgm-1A-1G or Cgm-2A-2E.

6. The composition of claim 1, configured as a topical, human skin probiotic moisturizer formulation further comprising a nutrient source for the bacteria, wherein the formulation is acidic to prevent skin colonization by other harmful bacteria.

7. The composition of claim 6, disposed on the skin of a person.

8. The composition of claim 6, wherein the nutrient source comprises one or more sugars.

9. The composition of claim 6, further comprising a humectant, such as glycerin, hyaluronic acid, and propylene glycol, or an emollient, such as shea butter, cocoa butter, and octyldodecanol, or an occlusive, such as petrolatum, cetyl alcohol (hexadecan-1-ol), and lanolin.

10. The composition of claim 6, engineered to produce a skin-bioactive agent, wherein the bacteria are producing the agent.

11. The composition of claim 10, wherein the bioactive agent is selected from a cosmetic, anti-aging, anti-oxidant, sun-screen, anti-inflammatory, antimicrobial, analgesic, and therapeutic compound.

12. The composition of claim 10, wherein the bioactive agent is selected from signal peptides, carrier peptides, neurotransmitter inhibitor peptides, and enzyme inhibitor peptides.

13. The composition of claim 10, wherein the bioactive agent is selected from lysine, arginine, cysteine, histidine, alanine, serine, theronine, isoleucine, aspartic acid, valine, citrulline, GHK-Cu, GSH-Cu, mangenese tripeptide, Peptamide-6, carnosine, N-acetyl carnosine, tripeptide-10-citrulline, palmitoyl-tripeptides, palmitoyl-tetrapeptides, palmitoyl-pentapeptides, acetyl-tetrapeptide, hexapeptide-11, tetrapeptide PKEK, hexapeptide-14, silk protein, aquaporin, alpha interferon, Hsp70, transforming growth factors, rice peptides, and soybean peptides.

14. The composition of claim 10, wherein the bioactive agent is a serine protease inhibitor, such as LEKTI-D6, LEKTI-D5, elafin and secretory leukocyte protease inhibitor for treating Netherton Syndrome, atopic dermatitis, psoriasis, and acne rosacea.

15. The composition of claim 10, wherein the bioactive agent is an anti-inflammatory compound, such as IL-10 to treat diseases such as Netherton Syndrome, atopic dermatitis, psoriasis, acne rosacea, and chronic wound infection.

16. The composition of claim 10, wherein the bioactive agent is an anti-microbial peptide, such as pediocin and nisin to treat diseases such as Netherton Syndrome, atopic dermatitis, psoriasis, acne rosacea, and chronic wound infection.

17. The composition of claim 10, wherein the bioactive agent is an antibody, antibody fragment or nanobody, such as single chain variable fragment anti-tumor necrosis factor alpha to treat diseases such as Netherton Syndrome, atopic dermatitis, psoriasis, acne rosacea, and chronic wound infection.

18. A method of using composition of claim 6, comprising applying the formulation to the skin of a person in need thereof under conditions wherein the bacteria produce an effective amount of the agent on the skin over a predetermined time range.

19. A method of claim 18, wherein the time range is 4, 8, 12, 24, and 48 hours.

20. A method of claim 18, further comprising adjusting the concentration of the nutrient source to adjust bioproduction and colonization time, wherein the concentration of glucose and glycerol is varied from 2 to 20% of the moisturizer for increasingly longer colonization times.
